(19) United States
(12) Patent Application Publication
(10) Pub. No.: US 2026/0216481 A1
(43) Pub. Date: Jul. 30, 2026

(54) SIDE-LOOKING EXPANDABLE FRAME AND DEVICE FOR STEERING INTERVENTIONAL DEVICES

(71) Applicant: MAGELLAN BIOMEDICAL INC, RICHMOND HILL (CA)

(72) Inventors: YARA ALAWNEH, RICHMOND HILL (CA); ALYKHAN SEWANI, RICHMOND HILL (CA); ANDREW DUECK, RICHMOND HILL (CA); MOHAMMAD TAVALLAEI, RICHMOND HILL (CA)

(21) Appl. No.: 19/147,178

(22) PCT Filed: Jan. 10, 2024
(86) PCT No.: PCT/US2024/011044

§ 371 Date: Jul. 10, 2025

Related U.S. Application Data
(60) Provisional application No. 63/438,187, filed on Jan. 10, 2023.

(51) Int. Cl. A61M 25/01; A61B 17/00; A61M 25/00; A61M 25/06

(52) U.S. Cl. CPC A61M 25/0152; A61M 25/0015; A61M 25/0074; A61M 25/0108; A61B 2017/00336; A61M 2025/0681

(57) ABSTRACT

A side-looking steering device for positioning an interventional device within a patient. The side-looking steering device includes an angled internal catheter, a side-looking expandable frame, and a sheath. The angled internal catheter is made of tubular flexible material and includes a distal end portion that is biased to bend to one side when unconstrained. The side-looking expandable frame includes a tubular base and a plurality of leaflets. The tubular base has a proximal end and a distal end with the angled internal catheter extending through the tubular base. The plurality of leaflets each have varied lengths and widths. Each leaflet, when unconstrained, extends from the distal end of the tubular base and is biased in shape such that it extends outwardly and in a bent orientation to one side to peripherally surround the distal tip of the angled internal catheter.

### CROSS-REFERENCE TO RELATED APPLICATIONS

The present application claims priority to U.S. Provisional Patent Application No. 63/438,187, filed on Jan. 10, 2023, which is fully incorporated herein by reference.

### TECHNICAL FIELD

The present disclosure relates to steering of interventional devices, such as guidewires, catheters, and needles. More particularly, the disclosure relates to devices with a side-looking expandable frame for guiding interventional procedures and related methods. Further, the disclosure relates to steering and tracking of interventional devices for medical procedures.

### BACKGROUND

Catheters and guidewires are the main interventional tools for minimally invasive procedures. They are long, flexible devices that can be inserted into the anatomy and are controlled remotely from outside the body. Guidewires are typically used to navigate through the anatomy while catheters run along the guidewire to provide support and allow for delivery of other interventional devices such as balloons, stents, leads, needles, and implants. Due to the limitations of remote control of these devices, most of the procedure time is dedicated to the maneuvering of catheters and guidewires.

Many cardiovascular procedures are performed with minimally invasive techniques using catheters and guidewires. Example applications may include therapeutic or diagnostic procedures, all of which require accurate manipulation of interventional devices to anatomical targets of interest.

To address limitations in navigation of guidewires, catheter tips are pre-shaped to various curvatures to help direct the guidewire in a particular direction. However, these still suffer from the same navigation and support limitations as straight catheters and can require cycling through multiple devices to reach a point of interest. Another method uses a cable, fixed to the side of the catheter tip that runs back to the handle and can be actuated to deflect the catheter. The catheter tip has a section made of softer material than the rest of the catheter to maximize the amount of deflection that occurs at the tip.

An additional method uses an external magnetic field to directly manipulate the catheter tip. This may be achieved by integrating magnets at the catheter tip and adjusting of the external magnetic field to control the device position. The field can be controlled remotely from outside the operating room, reducing user exposure time to ionizing radiation from the X-Ray image guidance. These magnetically actuated catheters typically require dedicated operating rooms for the equipment to generate magnetic fields, and custom catheters to be compatible with the technology. Mechanical limitations of catheter devices are exacerbated by the 2D guidance of X-Ray fluoroscopy. Additionally, X-ray does not provide much resolution or contrast when imaging soft tissue. Alternate imaging modalities can be used to supplement the image guidance provided by X-Ray and to provide other detailed information about tissue structure, composition, and function for diagnostic purposes.

Other attempts at addressing some of these limitations include robotically steered catheters. Robotic catheters use motorized pull wires to mechanically advance, retract, and rotate the catheter remotely from outside the body. Hence, robotic tendon-based devices still suffer from the same limitations of navigation and steering of conventional devices.

Therefore, there exists a need for devices, systems and methods that can provide improved visualization, navigation, and device support in the desired direction for the procedure particularly for challenging sharp angles in many minimally invasive procedures.

### SUMMARY

The present disclosure addresses the limitations in accurate maneuvering and precise steering of interventional devices within the vascular system, particularly for applications where sharp deflection angles are needed. Embodiments disclosed propose a more stable steering system that may reduce the incidence of vessel trauma and the number of catheter exchanges/manipulations, as well as allowing for access of interventional devices in tortuous vasculature and hard-to-reach sites. Disclosed systems include a side-looking expandable frame that can be deployed or anchored within a vessel lumen, a cardiac chamber, a stent graft, or any anatomical site of interest.

An embodiment includes a side-looking steering device for positioning an interventional device within a patient. The side-looking steering device includes an angled internal catheter, a side-looking expandable frame, and a sheath. The angled internal catheter is made of tubular flexible material and includes a distal end portion that is biased to bend to one side when unconstrained. The distal end portion commences at an unbent first location and terminates at a distal tip. The side-looking expandable frame includes a tubular base and a plurality of leaflets. The tubular base has a proximal end and a distal end with the angled internal catheter extending through the tubular base. The plurality of leaflets have varied lengths and widths. Each leaflet, when unconstrained, extends from the distal end of the tubular base and is biased in shape such that it extends outwardly and in a bent orientation to one side to peripherally surround the distal tip of the angled internal catheter. The sheath has an elongate tubular shape, into which the angled internal catheter and the side-looking expandable frame are sized for axial withdrawal and collapse into a compressed state.

An embodiment includes a side-looking steering device for positioning an interventional device within a patient. The side-looking steering device includes a side-looking expandable frame, a set of strings anchored on the distal ends of the side-looking expandable frame, and an angled internal catheter. The side-looking expandable frame is shaped to deploy outwardly within a cavity, such as a vessel lumen, a stent graft, or a cardiac chamber. The side-looking expandable frame further having a plurality of elongate leaflet members having a common bend direction and distal ends. The set of strings are anchored on the distal ends of the side-looking expandable frame. The angled internal catheter can be secured inside the side-looking expandable frame and supported by the set of strings which are connected to the distal end of the angled internal catheter. The angled internal catheter is manipulated by using the set of strings which have at least one degree of freedom to control the position of an interventional device.

An embodiment includes a side-looking expandable frame for positioning an interventional device within a patient. The side-looking expandable frame can include a tubular base and a plurality of leaflets. The tubular base has a proximal end and a distal end. The tubular base can be sized for an angled internal catheter to extend therethrough. The plurality of leaflets are of varied lengths and extend from the distal end of the tubular base. Each of the plurality of leaflets are biased to expand a first portion outwardly in a straight configuration and biased to expand a second portion outwardly at an angled configuration to a side.

In an embodiment, the side-looking expandable frame houses a flexible angled internal catheter that is designed to allow for passage of interventional devices such as a guidewire, a catheter, a microcatheter, an energy source, a laser, a needle, an intravascular ultrasound, or an angioscope.

In another embodiment, the side-looking expandable frame can steer the internal catheter using a magnetic or an electric field generator that may be connected to the branches of the frame. In another embodiment, the side-looking expandable frame may be mounted on a multi-lumen internal catheter to permit passage of multiple interventional devices at the same time. In another embodiment, the distal ends of the expandable frame may act as an anchor to a plurality of strings or cables that are affixed to the distal tip of the internal catheter on one end and connected to a handle on the other end. By actuating the strings from the handle end, the internal catheter can be steered with multiple degrees of freedom to allow for controlled and accurate positioning of the catheter tip in a plane facing the area or vasculature of interest. The present disclosure provides a more stable mechanical system to deploy stents, micro coils, balloon expandable stents, or any other interventional device in a less traumatic manner.

In another embodiment, the side-looking expandable frame may have radiopaque markers on its distal ends to allow for better visualization and orientation of the frame's position relative to the anatomy, under fluoroscopy. The side-looking expandable frame branches may extend beyond the distal end of the internal catheter to create an offset from the anatomy and prevent the catheter tip from interacting with the area of interest as it is moving.

In another embodiment, the side-looking expandable frame may form eyelets at the distal tip to allow for the actuation of the plurality of strings or cables with minimal friction, for cable-based control and use of the frame as anchors for the cables in a parallel cable driven mechanism.

In yet another embodiment, the present disclosure may be used in interventional, diagnostic, or drug perfusion procedures such as regenerative stem-cell therapy, blood clot dissolving drugs, chemotherapeutic agents, inject contrast, or used for biopsy in oncology applications. The present disclosure may also be used for imaging purposes.

In another embodiment, the internal catheter may be flexible, but not so flexible as to prevent the user from being able to transfer meaningful force through the device unless supported by a portion of the anatomy, such as a vessel wall.

Applicant has recognized a need for the present disclosure in various fields of interventional devices. In one example, a procedure may involve a fenestrated endovascular aortic repair procedure, a minimally invasive approach used to place an expandable fenestrated stent graft within the dilated aorta. An important step in this procedure is to gain access to vital arteries, that provide blood supply to other organs, from within the stent graft. Often, a J-tip catheter is used to direct a guidewire to the target site to facilitate vessel cannulation and catheterization. However, due to the extreme takeoff angle of targeted vessels from its parent artery, the level of steerability and torquability of these catheters and guidewires are rather low. Therefore, a side-looking expandable frame may reduce the likelihood of an increase in procedure time, radiation exposure, contrast volume, and the risk of renal failure.

Another example, where a guidewire is used to facilitate the placement of stent grafts in supplying arteries, is an endovascular aortic arch repair procedure. After the main graft is deployed, placement of branched grafts is required to allow for continued perfusion of the supra-aortic vessels. Although this treatment has become the standard of care, risk of stroke is still a major concern. Due to the curvature of the arch, distance from the femoral access vessels, and large pulsatile blood flow, access to the target-vessel ostia using conventional catheters requires a high degree of finesse and precise device manipulation. Use of a side-looking expandable frame allows the clinician to access the target-vessel ostia without extensive manipulation or re-situating of the catheter to accommodate the curvature of the arch.

In yet another example, catheter-based renal denervation procedures are used to treat patients with uncontrolled hypertension. Using an endovascular approach, a catheter is inserted in the renal arteries to disrupt renal-sympathetic nerves using radiofrequency ablation. Use of a side-looking expandable frame allows for acquiring adequate arterial wall contact in a tortuous and angled renal artery, where ablation is needed, leading to better improved outcomes.

As another example, mesenteric angioplasty and stent placement is a minimally invasive approach used to treat patients with chronic mesenteric ischemia. A hollow catheter is used to advance a guidewire through the plaque blocking the mesenteric artery. Excessive guidewire and catheter manipulations can increase the risk of branch perforation. Therefore, a side-looking expandable frame, using an angled catheter may allow for better placement, and less catheter-guidewire manipulation.

As another example, cardiac resynchronization therapy is used to treat patients with chronic heart failure. Using a transvenous approach, the coronary sinus is cannulated to allow for delivery of pacing leads into the coronary venous system. However, in most patients, accessing the coronary sinus remains a challenge due to difficulties that arise from its anatomy and the orientation of its ostium. In addition to that, positioning and orienting conventional non-steerable catheters in a dynamic cardiovascular environment is very challenging, due to the limitations mentioned previously. Therefore, the side-looking expandable frame may allow for easier positioning due to the enhanced steerability. Rotation of catheters and sheaths inside the vasculature or multiple catheter exchanges might result in arrhythmias and vessel damage, so avoiding excessive manipulation by using a reliable, steering device, is preferred.

In yet another example, a transseptal puncture is an approach used by electrophysiologists and interventional cardiologists to preform procedures that require access to the left atrium of the heart. To treat different cardiac conditions in the left atrium, a needle must be inserted from the right atrium through the fossa ovalis to the left atrium. To avoid any sudden or uncontrolled forward movement of the needle that might increase the risk of cardiac perforation, thrombus formation, and air embolism, the side-looking expandable frame may be used to position the needle.

In another example, after a successful transseptal puncture, a minimally invasive mitral valve repair procedure is performed to treat mitral valve regurgitation or stenosis. However, due to the location of the mitral valve and its large size, the delivery device must have a high degree of flexion to be able to navigate the transseptal puncture and then subsequently be positioned to face the mitral valve. Another technical challenge, due to previously mentioned limitations, is the ability to maneuver and steer the device in a dynamic environment. Providing a side-looking expandable frame as mentioned, may allow for increased steerability and maneuvering to navigate the transseptal puncture.

In yet another example, a transjugular intrahepatic portosystemic shunt is a procedure performed to treat patients with liver cirrhosis. Due to scar tissue blocking blood flow in the liver, pressure in the portal vein can increase and cause the veins to rupture. Using imaging guidance, a catheter is used to place a stent between the portal vein and the hepatic vein to relieve the pressure and restore blood flow. However, before placing the stent, the portal vein must be punctured. Due to poor visualization and catheter limitations, portal vein localization can be challenging, and multiple puncture attempts can increase the risk of bleeding.

In another example, for the treatment of intracranial aneurysms, an endovascular coiling procedure uses a catheter and a microcatheter to release platinum coils into the area of the aneurysm to prevent blood flow. However, due to the limited level of control and restriction of microcatheter movements within the aneurysm, the risk of perforation can increase. Placing the coils requires precise positioning, and use of a side-looking expandable frame may allow entrance to the area of the aneurysm at a proper angle, and without excessive manipulation.

Many minimally invasive imaging modalities suffer from the small space restrictions within surgical catheters and have decreased field of view (FOV) or resolution. Images are also obtained with respect to a floating reference frame within the anatomy, making it difficult to know the absolute location of targets of interest. Additionally, registration of a separate interventional tool to the obtained image for guidance is difficult, especially if the imaging catheter acts independently of the interventional tool, for example most commercial ultrasound catheters do not have an internal lumen to support insertion of a guidewire. The present disclosure remedies these limitations by providing a side-looking expandable frame with a sheath capable of delivering an angled internal catheter. Imaging may be improved by the enhanced steering capabilities of the disclosed device.

Ultrasound as an imaging modality can be used for tissue characterization through measurement of acoustic impedance and reflection coefficients, elastography of tissue to determine mechanical properties, visualization of tissue to determine shape, location of targets of interest for image guidance, and measurement of blood flow using doppler. As well as the mechanical properties measured by ultrasound, an electrical probe could be used to measure electrical properties of the tissue such as impedance, conductivity, and permittivity through the use of electrodes.

Optical imaging techniques can also be implemented into an imaging probe. Possible applications are spectroscopy at various wavelengths, miniaturized endoscopes, and optical coherence tomography (OCT). Laser sources can also be used for ablation or combined with an acoustic receiver for photoacoustic imaging. Another imaging probe, a nuclear activity detector, may be used to measure high energy radiation stemming from areas of tissue with high nuclear activity.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1A is a perspective view of a side-looking steering device with a side-looking expandable frame and an angled internal catheter, according to an embodiment.

FIG. 1B is a perspective view of a side-looking steering device with a side-looking expandable frame that allows one degree of freedom, according to an embodiment.

FIG. 1C is a perspective view of a side-looking steering device with a side-looking expandable frame as a steering device that allows for two degrees of freedom, according to an embodiment.

FIG. 1D is a perspective view of a side-looking steering device with a side-looking expandable frame and an angled internal catheter, according to an embodiment.

FIG. 1E is a perspective view of a side-looking steering device with a side-looking expandable frame and an angled internal catheter, according to an embodiment.

FIG. 2 is a perspective view of a side-looking steering device with a side-looking expandable frame with a handle attached, according to an embodiment.

FIG. 3 is a perspective view of a side-looking steering device with a side-looking expandable frame, according to an embodiment.

FIG. 4 is a perspective view of a side-looking steering device with a side-looking expandable frame with a mesh-like structure, according to an embodiment.

FIG. 5 is a perspective view of a side-looking steering device with a side-looking expandable frame sutured, sewn, or glued onto a fabric, according to an embodiment.

FIG. 6 is a schematic view of use and deployment of a side-looking expandable frame in a FEVAR (Fenestrated Endovascular Aortic Aneurysm Repair) procedure to cannulate target vessels according to an embodiment.

FIG. 7 is a schematic view of use and deployment of a side-looking expandable frame for an aortic arch repair procedure to facilitate guidewire navigation from within the stent graft, according to an embodiment.

FIG. 8 is a schematic view of use and deployment of a side-looking expandable frame for renal denervation procedure to facilitate ablation of nerves in the renal artery, according to an embodiment.

FIG. 9 is a schematic view of use and deployment of a side-looking expandable frame for a superior mesenteric artery angioplasty and stenting procedure to facilitate guidewire navigation through the plaque and allow for stent graft ballooning, according to an embodiment.

FIG. 10 is a schematic view of use and deployment of a side-looking expandable frame for a coronary angioplasty procedure in order to facilitate guidewire navigation for coronary artery cannulation, according to an embodiment.

FIG. 11 is a schematic view of use and deployment of a side-looking expandable frame for cardiac resynchronization therapy to facilitate placement of leads through the coronary sinus, according to an embodiment.

FIG. 12 is a schematic view of use and deployment of a side-looking steering device for needle navigation in a transseptal puncture, according to an embodiment.

FIG. 13 is a schematic view of use and deployment of a side-looking expandable frame for guidewire navigation in a transcatheter mitral valve repair procedure, according to an embodiment.

FIG. 14 is a schematic view of use and deployment of a side-looking expandable frame for guidewire navigation in a transcatheter tricuspid valve repair procedure, according to an embodiment.

FIG. 15 is a schematic view of use and deployment of a side-looking expandable frame for vein puncture and stent graft deployment in a transjugular intrahepatic portosystemic shunt procedure, according to an embodiment.

FIG. 16 is a schematic view of use and deployment of a side-looking expandable frame for delivery of coils in a brain, or heart aneurysm during an endovascular coiling procedure, according to an embodiment.

FIGS. 17A and B are perspective views of a side-looking expandable frame depicting stages of the manufacturing process, before and after expanding a side-looking expandable frame, according to an embodiment.

FIG. 18 is a perspective view of a sequence of expansion, of a side-looking expandable frame, according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Disclosed herein are devices, systems, and methods for steering a side-looking device, including guidewires or catheters, during various medical intervention procedures. Examples disclosed propose a more stable steering system that may reduce the incidence of vessel trauma and the number of catheter exchanges/manipulations. Embodiments of the disclosed device may also allow for access of interventional devices in tortuous vasculature and hard-to-reach sites. The disclosed device may include a side-looking expandable frame that can be deployed or anchored within a vessel lumen, a cardiac chamber, a stent graft, or any anatomical site of interest. The side-looking expandable frame may house a flexible bent or angled internal catheter that is designed to allow for passage of interventional devices such as a guidewire, a catheter, a microcatheter, an energy source, a laser, a needle, an intravascular ultrasound, or an angioscope.

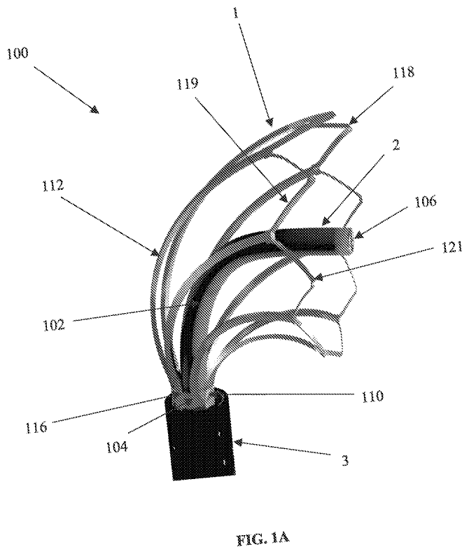

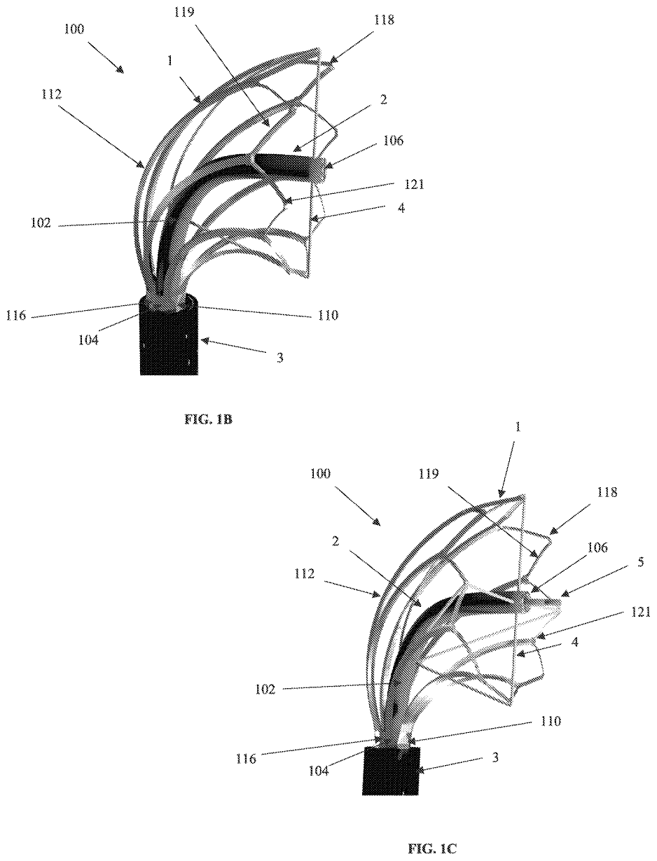

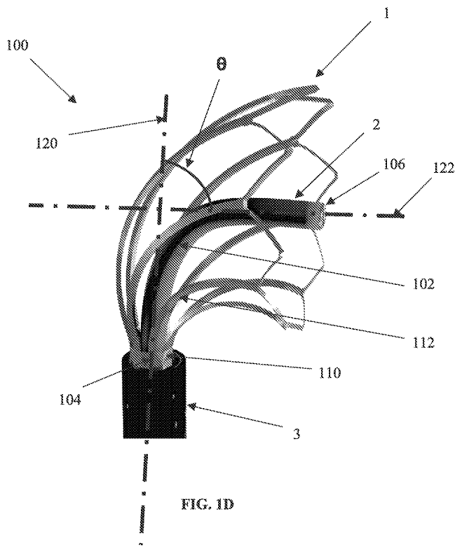

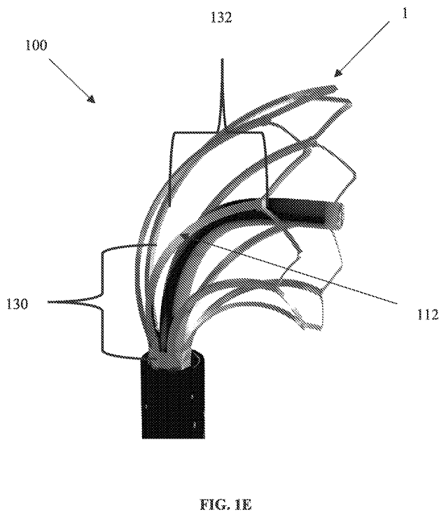

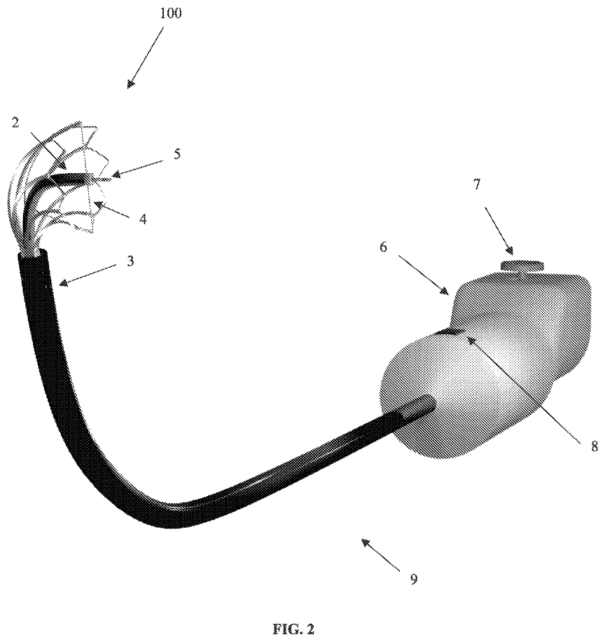

FIGS. 1A-1E disclose a side-looking steering device 100 for positioning an interventional device within a patient. The side-looking steering device 100 shown includes a side looking expandable frame 1, an angled internal catheter 2, and a sheath 3. Some embodiments of the side-looking steering device 100 further depict strings or cables 4 for directing a guidewire 5 or other interventional device.

Centrally located within FIGS. 1A-1E is an angled internal catheter 2. The angled internal catheter 2 is made of tubular flexible material and includes a distal end portion 102 that is biased to bend to one side when unconstrained. The distal end portion 102 commences at an unbent first location 104 and terminates at a distal tip 106. Accordingly, the distal end portion 102 is the portion of the angled internal catheter 2 that can be seen in FIGS. 1A-1E, for example.

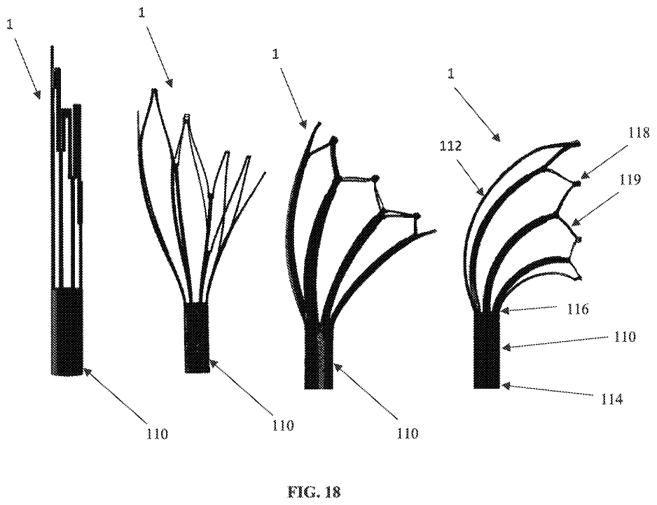

The side-looking expandable frame 1 includes a tubular base 110 and a plurality of leaflets 112. Specifically, eight leaflets 112 can be seen in FIG. 1A. The leaflets 112 shown are each elongate members having a common bend direction and distal end 116. This common bend direction referring to a similar overall, general direction and disposition of bend location, as shown in the figures. The leaflets having a common bend direction should not be interpreted so narrowly as to be limiting of these leaflets to structures of precisely the exact same shape, contour, and orientation. Tubular base 110 has a proximal end 114 and a distal end 116. The proximal end 114 and tubular base 110 cannot be seen well in FIGS. 1A-1E, but see FIG. 18, for example. In FIGS. 1A-1E, the angled internal catheter 2 is shown extending through the tubular base 110. The plurality of leaflets 112 have varied lengths and widths. Each leaflet 112, when unconstrained as shown, extends from the distal end 116 of the tubular base 110. Each one of the leaflets 112 is biased in shape such that it extends outwardly and in a bent orientation to one side to peripherally surround the distal tip 106 of the angled internal catheter 2 with the distal ends 118 of the leaflets 112. The side-looking expandable frame 1 and its leaflets 112 can be formed from a tube in certain embodiments. In some embodiments, the side-looking expandable frame 1 is formed by laser cutting, photoetching, Electric Discharge Machining (EDM), or by water jet abrasion. In some embodiments, the side-looking expandable frame 1 includes a shape memory metal. Such a shape memory metal may include a nickel titanium alloy, for example.

In some embodiments, the leaflets 112 are joined by at least one strut 119. In some embodiments, the structure of at least one strut 119 is varied along its length. In some embodiments, at least one strut 119 has a plurality of connection points 121 which allows for joining the plurality of leaflets 112.

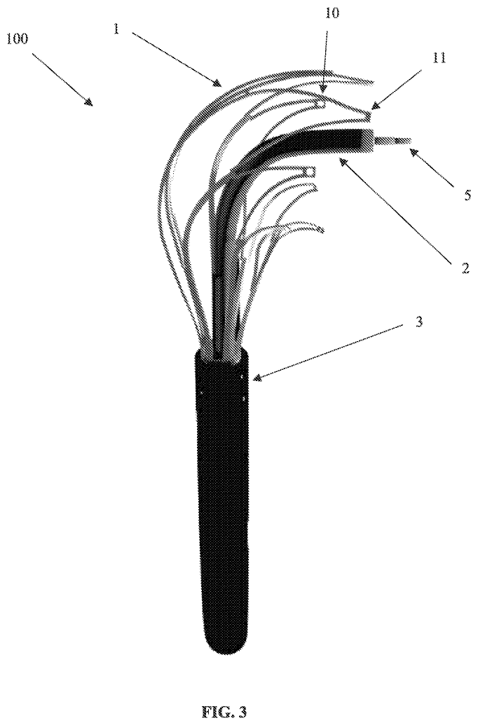

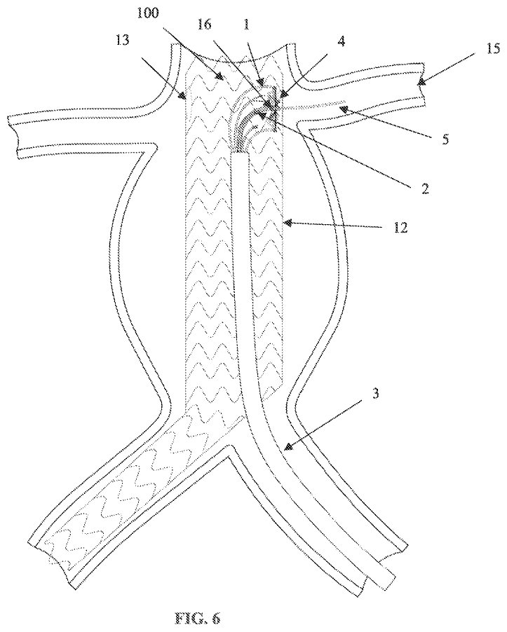

In some embodiments, a set of strings 4 is anchored to the distal ends 118 of the leaflets 112 of the side-looking expandable frame 1. See FIGS. 1B and 1C, for example. In some embodiments, leaflets 112 may extend to distal ends 118 where apertures 10 are present that form an eyelet configuration (see FIG. 3, for example). In some instances, the leaflets 112 may extend to distal ends 118 with radiopaque markers 16 (See FIG. 6, for example).

Although only partially shown in FIGS. 1A-1E, the sheath 3 may generally be of an elongate tubular shape. Into this tubular shape, the angled internal catheter 2 and side-looking extendable frame 1 are sized for axial withdrawal. Specifically, they are withdrawn and collapsed into a compressed state.

The side-looking, curved and angled nature of the device 100 and frame 1 can be understood in various ways. FIG. 1D illustrates one example, in which the distal end portion 102 of the angled internal catheter 2 has a first tubular axis 120 at an unbent first location 104 and the distal end portion 102 of the angled internal catheter 2 has a second tubular axis 122 at the distal tip 106. In some side-looking device embodiments, the angle θ between the first tubular axis 120 and the second tubular axis 122 is at least 50 degrees. In some side-looking device embodiments, the angle θ between the first tubular axis 120 and the second tubular axis 122 is between 70 and 110 degrees. In some side-looking device embodiments, the angle θ between the first tubular axis 120 and the second tubular axis 122 is approximately perpendicular.

In some embodiments, such as in FIG. 1C, the side-looking steering device 100 can be understood to include a side-looking expandable frame 1, a set of strings 4 anchored on the distal end of the side-looking expandable frame 1, and an angled internal catheter 2. The side-looking expandable frame 1 is shaped to deploy outwardly within a cavity, such as a vessel lumen, a stent graft, or a cardiac chamber, the side-looking expandable frame having a distal end. The angled internal catheter 2 can be secured inside the side-looking expandable frame 1 and supported by the set of strings 4 which are connected to a distal end 102 of the angled internal catheter 2. The angled internal catheter 2 is manipulated by using the set of strings 4 which have at least one degree of freedom to control the position of an interventional device.

The side-looking expandable frame 1 can be retracted inside the sheath 3 and redeployed while maintaining its maximum expanding diameter. The side-looking expandable frame 1 can maintain its rigidity when the angled internal catheter 2 is steered. In some embodiments, the angled internal catheter 2 is bent at a 90-degree angle. The side-looking expandable frame 1 can include multiple leaflets 112, and the leaflets 112 are joined by multiple struts 119.

In some embodiments, a side-looking expandable frame 1 can include a tubular base 110 and a plurality of leaflets 112. The tubular base 110 has a proximal end 114 and a distal end 116. The tubular base 110 can be sized for angled internal catheter 2 to extend therethrough. The plurality of leaflets 112 are of varied lengths and extend from the distal end 116 of the tubular base 110.

As shown in FIG. 1E, each of the plurality of leaflets 112 can be biased to expand a first portion 130 outwardly in a straight configuration and biased to expand a second portion 132 outwardly at an angled configuration to a side. In some embodiments, the angled configuration of the second portion 132 differs from the straight configuration of the first portion 130 by at least 50 degrees. As seen in FIG. 1E, the plurality of leaflets 112 each vary in width.

Further, the distal end 118 of the plurality of leaflets 112 can form aperture 10 with an eyelet configuration. In some embodiments, the plurality of leaflets 112 have distal ends 118 with radiopaque markers 16. In some embodiments, the plurality of leaflets 112 have at least one strut 119 and can include a plurality of connection points 121, which allow for joining of the plurality of leaflets 112.

Referring now specifically to FIG. 1A, a side-looking expandable frame 1 may be mounted on an angled internal catheter 2. The angled internal catheter 2 may be designed to allow for passage of interventional devices such as a guidewire, a catheter, a microcatheter, an energy source, a laser, a needle, an intravascular ultrasound, or an angioscope. The side looking expandable frame 1 may be collapsed inside a hollow cylindrical tube or a sheath 3. The hollow cylindrical tube or sheath 3 may vary in length and width, depending on the interventional procedure that is being completed.

In one example, the angled internal catheter 2 may be used to facilitate cardiac resynchronization therapy and treat patients with chronic heart failure. Using a transvenous approach, the coronary sinus is cannulated to allow for delivery of pacing leads into the coronary venous system. The side-looking expandable frame 1 facilitates access to the coronary sinus, and improves orientation in terms of accessing its ostium. In addition, positioning the angled internal catheter 2 becomes less challenging. Therefore, the side-looking expandable frame 1 allows for easier positioning and additional steering due to the curved orientation of the side-looking expandable frame.

Referring now to FIGS. 1B and 1C, perspective views of a side-looking expandable frame 1 that allows for one, or two degrees of freedom, respectfully, housing an angled internal catheter 2 that may allow for passage of interventional devices such as a guidewire 5. The side-looking expandable frame 1, may also act as an anchor to a plurality of strings or cables 4 and may be retracted inside a cylindrical tube or sheath 3.

Referring now to the embodiment in FIG. 2, a steering device 9 may include a side-looking expandable frame 1 positioned inside of a sheath 3. The sheath 3 may be used to steer an angled internal catheter 2 by moving the joystick 7 found on a handle side 6. The joystick 7 may be configured to actuate a plurality of strings or cables 4 to allow for steering of the angled internal catheter 2 in two degrees of freedom. Alternatively, and as seen in FIG. 1B, the angled internal catheter 2 may be steered in one degree of freedom. A guidewire 5 may then be passed through a lumen of the angled internal catheter 2. The handle side 6 may have a sliding button 8 that allows for tension on the plurality of strings or cables 4 to be released.

In examples, by actuating the plurality of strings or cables from the handle side 6, the angled internal catheter 2 may be steered with multiple degrees of freedom to allow for controlled and accurate positioning of the catheter tip in a plane facing the area or vasculature of interest. The present disclosure provides a more stable mechanical system to deploy stents, micro coils, balloon expandable stents, or any other interventional device in a less traumatic manner.

Referring now to FIG. 3, a perspective view of an alternative design of the side-looking expandable frame 1, which may be mounted on a sheath 3 and used as a steering device 9, according to an example. The side-looking expandable frame 1 may have one or more apertures 10 containing holes on a distal tip for the plurality of strings or cables 4 to pass through and anchor on. In another example, the side-looking expandable frame may also have extensions 11 on the distal ends to prevent the angled internal catheter 2 from interacting with anatomy that the side-looking expandable frame 1 is deployed in or anchored to.

In various embodiments, the side-looking expandable frame 1, may be formed from a tube. In alternative or additional embodiments, the side-looking expandable frame 1 may be formed by laser cutting, photoetching, EDM, or by water jet abrasion. In various embodiments, the side-looking expandable frame may include a shape memory metal. The shape memory metal can comprise a nickel titanium alloy, for example. As shown in FIG. 18, the portions of the side-looking expandable frame 1 that have been cut from a tube can be understood from its first depiction of a compressed state. Further the continuous, one-piece structure of the side-looking expandable frame 1 is shown.

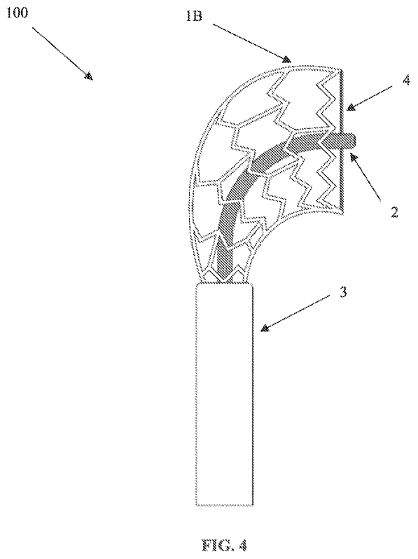

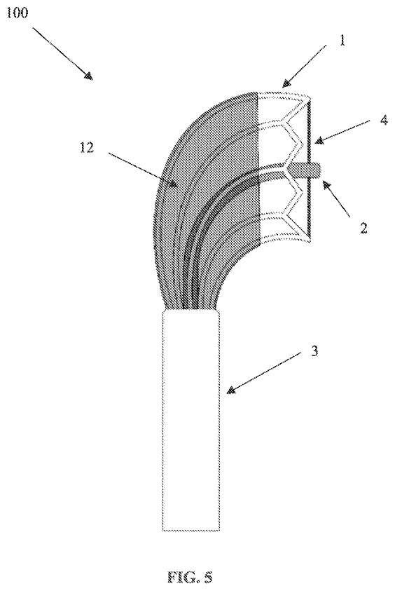

Referring now to FIG. 4, the side-looking expandable frame may have a mesh basket 1B that is shape set to the side. The plurality of strings or cables 4 anchored on the basket 1B are used to steer the angled internal catheter 2. The angled internal catheter 2 may be deployed from or retracted into a sheath 3. The sheath 3 may vary in length and design based on the intended interventional medical procedure.

Referring now to FIG. 5, an example of a side-looking expandable frame 1 with a fabric 12 sewn, sutured, or glued on an outer or inner surface of the side-looking expandable frame 1 to add additional mechanical support or join multiple branches together. The fabric 12 allows the frame 1 to maintain its rigidity when the plurality of strings or cables 4 are actuated to steer the angled internal catheter 2 during an interventional procedure.

Referring now to FIG. 6, the side-looking expandable frame may be used for a procedure to target vessel cannulation, according to one example. The side-looking expandable frame 1 may be deployed from inside the sheath 3, to be placed within a stent graft 13 with fenestrations or openings 14. Radiopaque markers 16 may be used and placed on distal ends of the side-looking expandable frame 1 and the angled internal catheter 2.

In one example, after confirming that the side-looking expandable frame 1 is facing the ostia of the renal artery 15, the plurality of strings or cables 4 may then be manipulated to steer the angled internal catheter 2 to a position facing the fenestrations or openings 14. A guidewire 5 may then be passed through the lumen of the angled internal catheter 2 to perform cannulation and catheterization of the renal artery 15.

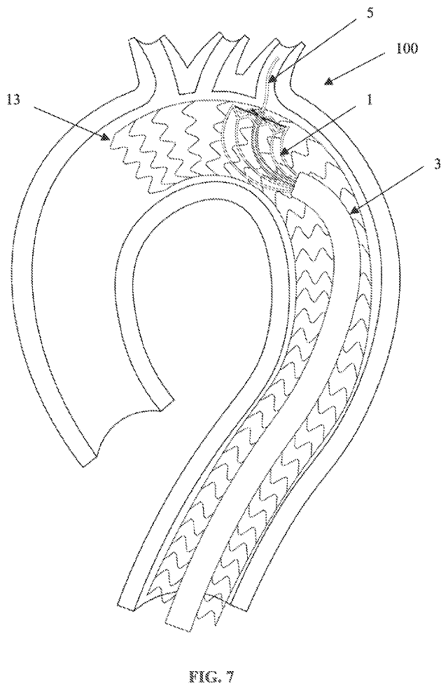

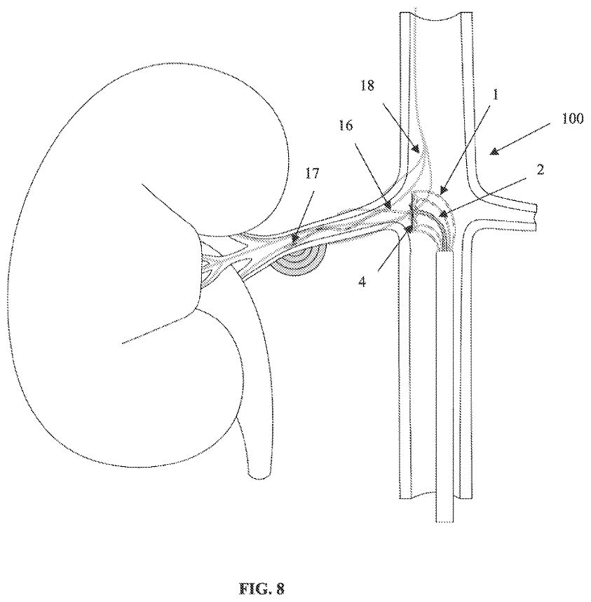

Referring now to FIG. 7, an illustration of the side-looking expandable frame 1 being used in an aortic arch repair procedure. The side-looking feature of the side-looking expandable frame 1 gives it an additional mechanical advantage when performing target vessel cannulation using a guidewire 5 from inside a stent graft 13. When the guidewire is deployed from within the sheath 3, the guidewire 5 is constructed to orient itself in the same plane as the ostium to be treated.

Referring now to FIG. 8, the side-looking expandable frame 1 may be positioned to face the ostia of the renal artery 15 to pass an ablation catheter 16 through the lumen of the angled internal catheter 2. Manipulation of the plurality of strings or cables 4 may be manipulated to steer the angled internal catheter 2, and in return steer the ablation catheter 16 to different positions to make sure that the radiopaque markers 17 on the ablation catheter 16 are in contact with nerves 18 to be ablated.

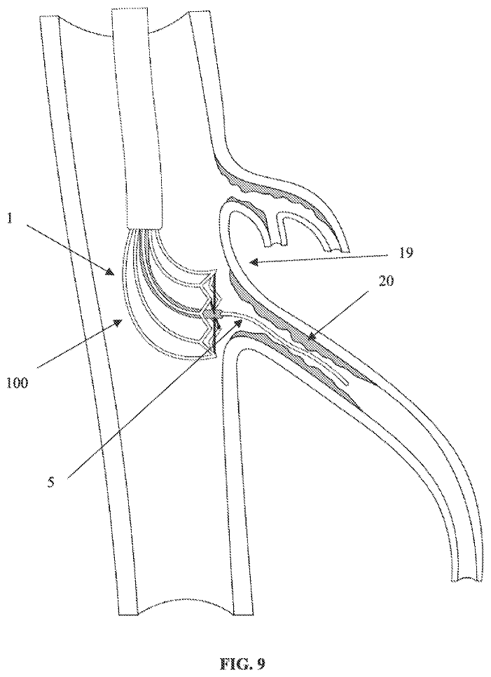

Referring now to FIG. 9, an exploded view of the side-looking expandable frame 1 positioned inside the superior mesenteric artery 19 to facilitate navigation of a guidewire 5 through plaque 20 for the purpose of performing an angioplasty procedure. The guidewire 5 may be deployed at an angle to properly enter the superior mesenteric artery 19. The side-looking expandable frame 1 allows for the guidewire 5 to enter the superior mesenteric artery 19, through the plaque 20, without repositioning the steering device.

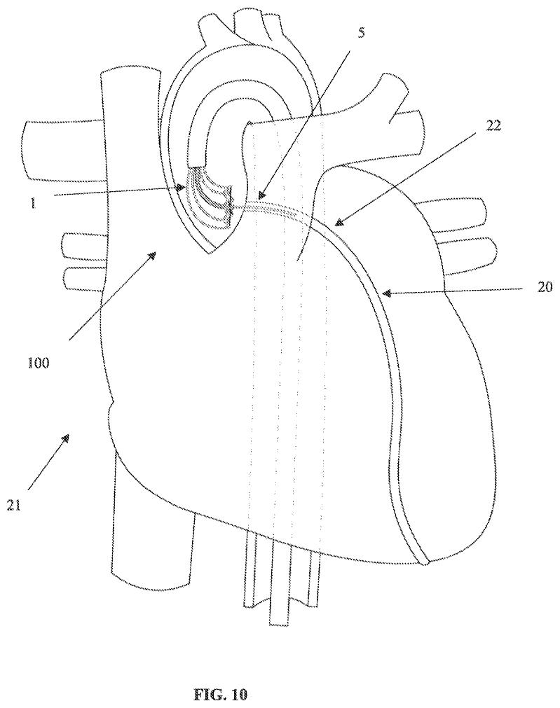

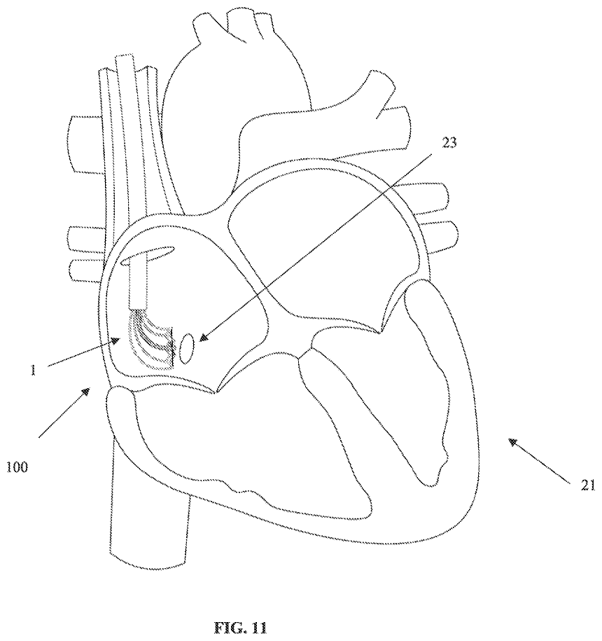

Referring now to, FIG. 10, the side-looking expandable frame 1 may be deployed inside the heart 21 and facing the coronary artery 22. The angle of the side-looking expandable frame 1 may facilitate precise navigation of a guidewire 5 through the plaque 20 as part of an angioplasty and stent placement procedure. A mesenteric angioplasty and stent placement is a minimally invasive approach used to treat patients with chronic mesenteric ischemia. The angled internal catheter 2 may be used to advance the guidewire 5 through the plaque 20 blocking the mesenteric artery. Excessive guidewire and catheter manipulations can increase the risk of branch perforation. Therefore, a side-looking expandable frame 1, using an angled catheter 2 may allow for better placement, and less catheter-guidewire manipulation. In another example, the side-looking expandable frame 1 may enter through additional vessels to access different chambers and vessels of the heart.

Referring now to FIG. 11, another example of the side-looking expandable frame 1 being deployed in the heart 21, facing the coronary sinus 23, to facilitate accurate placement of pacing leads in the coronary venous system. Cardiac resynchronization therapy is used to treat patients with chronic heart failure. Using a transvenous approach, the coronary sinus 23 is cannulated to allow for delivery of pacing leads into the coronary venous system. However, in most patients, accessing the coronary sinus remains a challenge due to difficulties that arise from its anatomy and the orientation of its ostium. Therefore, the side-looking expandable frame 1 may allow for easier positioning due to its curved shape. Rotation of catheters and sheaths inside the vasculature or multiple catheter exchanges might result in arrhythmias and vessel damage, so use of an angled internal catheter 2, delivered through the sheath 3, may lead to improved procedure outcomes.

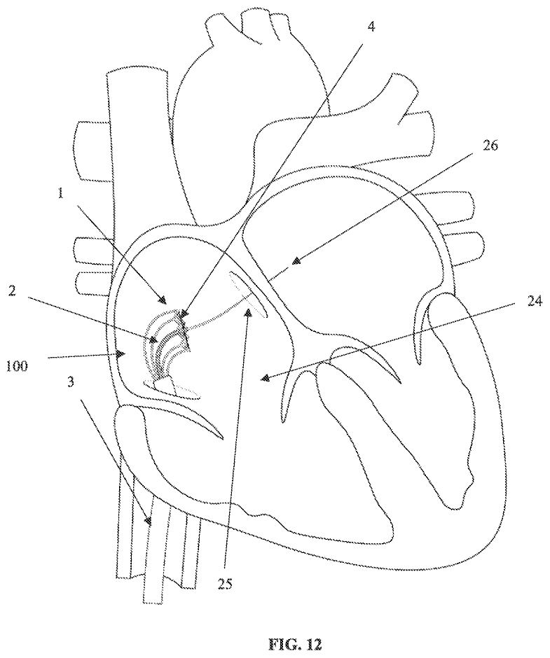

Referring now to FIG. 12, the side-looking expandable frame 1 may be positioned in the right atrium 24 when advanced out of the sheath 3. The plurality of strings or cables 4 may be actuated to precisely steer the angled internal catheter 2 to face the fossa ovalis 25 and allow the passage of a needle 26 to preform a transseptal puncture. The angled internal catheter 2 may be deployed from the sheath 3, which may be provided in multiple lengths to facilitate different anatomy in different users.

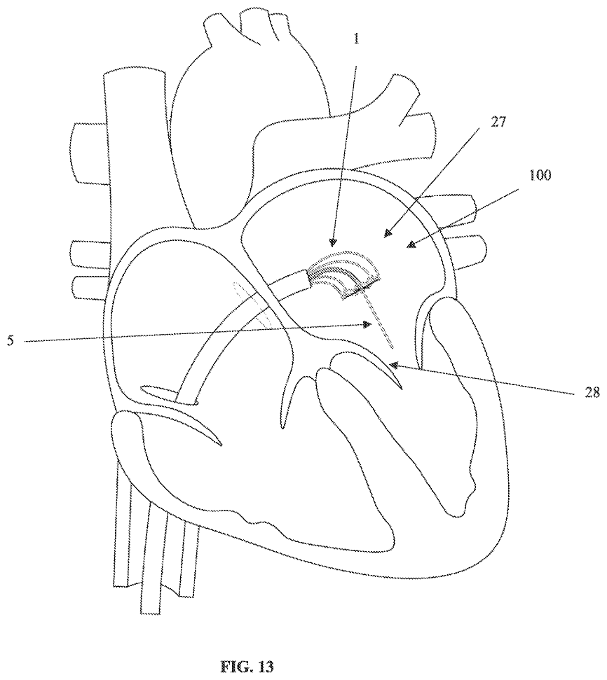

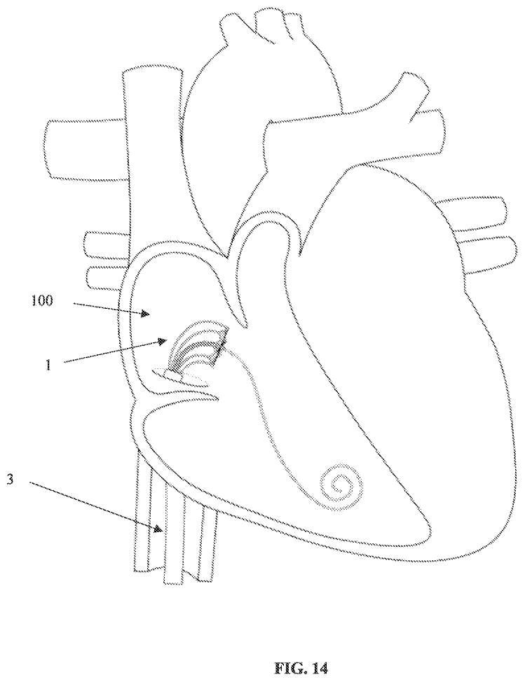

In another example, and as seen in FIG. 13, the side-looking expandable frame 1 may be deployed in the left atrium 27 to facilitate the navigation of a guidewire 5 through the mitral valve 28. The guidewire 5 may be inserted into the mitral valve 28 of the left atrium 27 as part of a mitral valve repair procedure. Mitral valve 28 repairs are often required after a successful transseptal puncture, to treat mitral valve regurgitation or stenosis. Due to the location of the mitral valve 28 and its large size, the side-looking expandable frame 1 may allow for increased flexion to navigate the mitral valve. Additionally, the expandable nature of the side-looking expandable frame 1 allows for increased precision and manipulation by the clinician, to address differing anatomy in different patients.

In another example, FIG. 14 illustrates the use and deployment of the side-looking expandable frame 1 in a tricuspid valve repair procedure. The sheath 3 may be inserted into the right atrium of the heart, to reach the tricuspid valve.

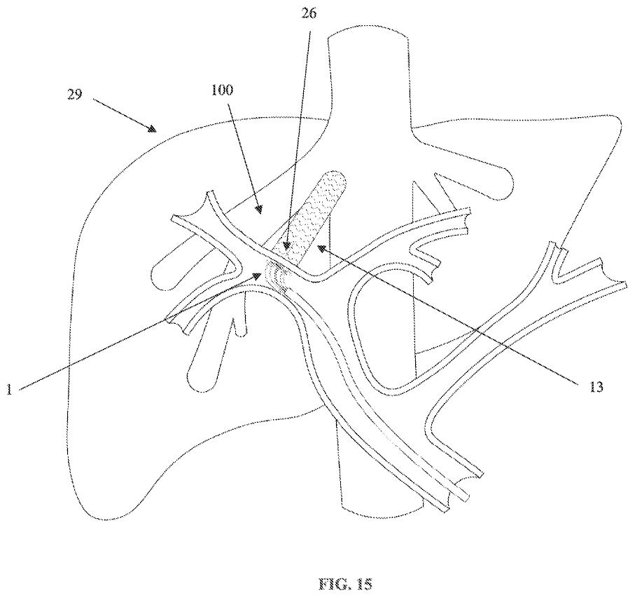

Referring now to, FIG. 15 an example of an application of the side-looking expandable frame 1, where the side-looking expandable frame 1 is deployed inside the liver 29. Once deployed, the side-looking expandable frame 1 may deliver a needle 26 to perform a hepatic artery puncture. A stent graft 13 may then placed through the puncture site, as part of a transjuagular intrahepatic portosystemic shunt procedure. In another example, a guidewire 5 may be used for interventional procedures within the liver, rather than a needle 26. Depending on the clinical need, the side-looking expandable frame 1 may deploy various tools and objects through the sheath 3 for different medical purposes.

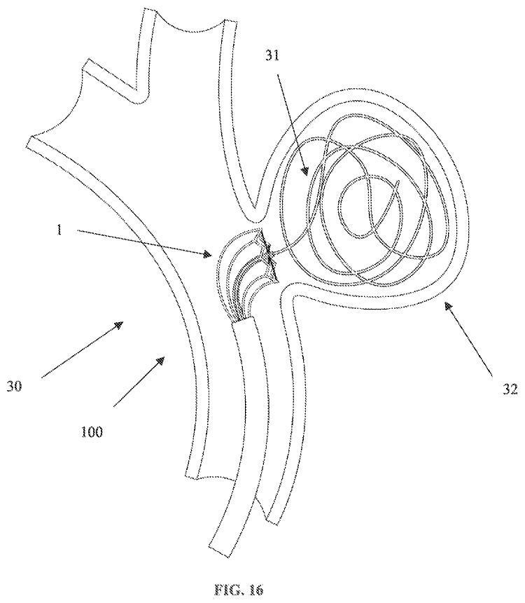

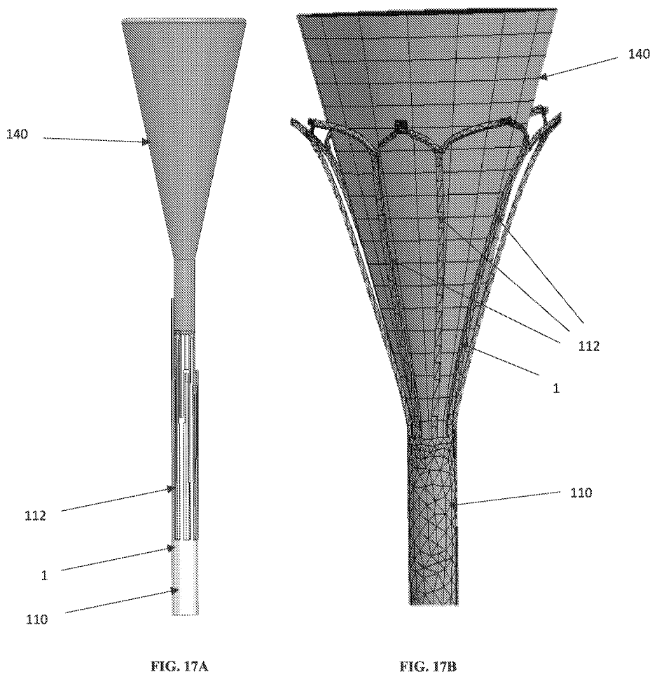

Referring now to FIG. 16, the side-looking expandable frame 1 may be deployed inside a cerebral artery 30. Once deployed, the side-looking expandable frame 1 may facilitate delivery of coils 31 inside an aneurysm 32, as part of an endovascular coiling procedure. The side-looking expandable frame 1 is configured to deploy at an angle adjusted to deliver the coils 31 precisely.

FIGS. 17A, 17B, and 18 depict aspects of manufacturing processes that can be used to produce a side-looking expandable frame 1, as described throughout the embodiments of this disclosure. The side-looking expandable frame 1 may fabricated from a tubular body (a cylindrical tube) made out of a superelastic or shape memory alloy, such as nitinol. The method of creating the side-looking expandable frame 1, may include forming a stent-cut or a pattern by selective removal of the material by means of laser-cutting, water jet cutting, chemical milling, or electrical discharge machining.

After the removal process, the shape of the cut tube may be asymmetric with one side longer than the other, to permit deforming of the material and obtaining the desired side-looking shape. The start of the shape-setting process includes a series of incremental steps that utilize custom-designed molds of different sizes to expand the cut nitinol tube from its initial diameter to its fixture diameter by heating it at elevated temperatures. Once set and cooled, it is reheated to go through a second shape setting process that deforms it to the side by using fixtures of different shapes and bending angles.

The embodiments shown in FIGS. 17A and 17B, respectively depict certain early stages where a side-looking expandable frame 1 is cut or machined in a collapsed configuration and a second stage of expanding the side-looking expandable frame 1 with incremental custom molds 140. This manufacturing process includes expanding the initially tubular cut tube with a variety of heating, cooling, reheating and deformation steps. FIG. 18 further depicts the stages of curvature and expansion of leaflets 112 that must be manufactured for reliable deformation when the side looking expandable frame is unconstrained within a patient's body.

FIGS. 17A and 17B further provide an example view of a side-looking expandable frame 1 before, and after expanding, according to one example. The side-looking expandable frame 1 may start in a closed, or constricted position to allow access to smaller anatomy. The sheath 2 may very in length and width, depending on the procedure to be completed, and what tools may be delivered or positioned through the sheath. After being inserted or maneuvered into the targeted anatomy, the side-looking expandable frame 1 may be expanded or spread as seen in FIG. 17B. The expansion may allow for easier anchoring of devices, more precise placement, and ease of delivery.

In embodiments, the side-looking expandable frame 1 may have a tubular body with a proximal end and a distal end, and a plurality of leaflets. Each leaflet may be affixed to the distal end of the tubular body of the shaft 2. Further, the leaflets may have a first portion and a second portion, each expanding in a different configuration. For example, the first portion of the leaflets may gradually expand in a straight configuration, while the second portion of the leaflets may be deformed to expand to the side at an angle, with the distal tips of the leaflets bent inwards. In alternative examples, the leaflets may expand in reversed configurations, or additional configurations as deemed appropriate or necessary by the procedure being completed.

In embodiments, the leaflets may vary in length and width, depending on the minimally invasive procedure being completed. The distal ends of the leaflets may also form an eyelet configuration, and/or have radiopaque markers. The leaflets have at least one strut, and the strut may vary in width along the length of the strut. The at least one strut may have a plurality of connection points, which allow for joining of the plurality of leaflets.

Referring now to FIG. 18, a sequence of expansion, of an expandable frame, according to one example. The side-looking expandable frame 1, may expand to various degrees as seen in FIG. 18. At each level of expansion, the growth or spreading of the side-looking expandable frame 1 may be stopped, or paused, to allow increased usability of, and precise placement of devices and therapies being delivered. Further, the side-looking expandable frame 1 may be controlled to spread out gradually within a human vessel or organ and configured to deploy. For example, if the targeted anatomy exists in a small, or difficult to reach area, the side-looking expandable frame 1 may only expand partially, allowing for increased access to, and better delivery of therapies and devices.

In assembly, the present disclosure proposed describes a self-expanding side-looking frame 1 that can be collapsed into a compressed configuration by means of a delivery sheath 3 then radially expanded at the treatment site when the sheath or delivery device is retracted.

In operation, the side-looking expandable frame 1 may be retracted inside of the sheath 3 and redeployed while maintaining its maximum expanded diameter. The side-looking expandable frame may include a cable-driven mechanism for positioning an interventional device. As depicted in various figures, such an interventional device may be located at the distal tip 106 of the angled internal catheter 2. Once the side-looking expandable frame 1 has been deployed within a cavity, such as a vessel lumen, a stent graft, or a cardiac chamber, the plurality of strings or cables 4 may then be anchored on a distal end of the side-looking expandable frame 1. The bent or angled internal catheter 2 is then secured inside the side-looking expandable frame 1 and is supported by the plurality of strings or cables 4, which are connected to the distal end of the angled internal catheter 2.

In examples, by using the plurality of strings or cables 4, the bent internal catheter 2 may be manipulated with at least one degree of freedom to control the position of the interventional device within the vessel lumen, the stent graft, or the cardiac chamber. The side-looking expandable frame 1 may maintain it's rigidity when the bent internal catheter 2 is steered. The bent internal catheter 2 may be bent or angled at a variety of angles, including a 90-degree angle. The plurality of strings or cables 4, may include a set of strings or cables of any number

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations, and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112 (f) are not to be invoked unless the specific terms “means for” or “step for” are recited in a claim.

1. A side-looking steering device for positioning an interventional device within a patient comprising:
an angled internal catheter of tubular flexible material, including a distal end portion that is biased to bend to one side when unconstrained, the distal end portion commencing at an unbent first location and terminating at a distal tip;
a side-looking expandable frame, including:
a tubular base having a proximal end and a distal end, the angled internal catheter extending through the tubular base;
a plurality of leaflets of varied lengths and widths, each leaflet when unconstrained extending from the distal end of the tubular base and biased in shape to extend outwardly and in a bent orientation to one side to peripherally surround the distal tip of the angled internal catheter; and
a sheath of elongate tubular shape into which the angled internal catheter and the side-looking expandable frame are sized for axial withdrawal and collapse into a compressed state.

2. The side-looking steering device of claim 1, wherein the side-looking expandable frame including the plurality of leaflets of varied lengths and width has been cut from a tube.

3. The side-looking steering device of claim 2, wherein the side-looking expandable frame is a continuous structure.

4. The side-looking steering device of claim 1, wherein the side-looking expandable frame is formed by laser cutting, photoetching, EDM, or by water jet abrasion.

5. The side-looking steering device of claim 1, wherein the distal end portion has a first tubular axis at the unbent first location and has a second tubular axis at the distal tip, when unconstrained.

6. The side-looking steering device of claim 5, wherein the angle between the first tubular axial orientation and the second tubular axial orientation is at least 50 degrees.

7. The side-looking steering device of claim 5, wherein the angle between the first tubular axial orientation and the second tubular axial orientation is between 70 and 110 degrees.

8. The side-looking steering device of claim 5, wherein the angle between the first tubular axial orientation and the second tubular axial orientation is approximately perpendicular.

9. The side-looking steering device of claim 1, wherein distal ends of the leaflets form an eyelet configuration.

10. The side-looking steering device of claim 1, wherein the leaflets have distal ends with radiopaque markers.

11. The side-looking steering device of claim 1, wherein the leaflets are joined by at least one strut.

12. The side-looking steering device of claim 11, wherein width of the at least one strut is varied along a length of the strut.

13. The side-looking steering device of claim 11, wherein the at least one strut has a plurality of connection points, which allow for joining of the plurality of leaflets.

14. The side-looking steering device of claim 1, further including a set of strings anchored on the distal end of the side-looking expandable frame providing a portion of a cable-driven mechanism for positioning an interventional device located at the distal tip of the angled internal catheter.

15. The side-looking steering device of claim 14, wherein the distal tip of the angled internal catheter is in planar alignment with anchoring locations of the set of strings on the distal end of the side-looking expandable frame.

16. A side-looking steering device for positioning an interventional device within a patient comprising:
A side-looking expandable frame that is shaped to deploy outwardly within a cavity, such as a vessel lumen, a stent graft, or a cardiac chamber, the side-looking expandable frame having a plurality of elongate leaflet members having a common bend direction and distal ends;
a set of strings anchored on the distal ends of the side-looking expandable frame;
an angled internal catheter that is secured inside the side-looking expandable frame and is supported by the set of strings which are connected to a distal end of the angled internal catheter;
wherein by using the set of strings, the angled internal catheter is manipulated with at least one degree of freedom to control the position of an interventional device.

17. The side-looking steering device of claim 16, wherein the side-looking expandable frame can be retracted inside a sheath and redeployed while maintaining its maximum expanding diameter.

18. The side-looking steering device of claim 16, wherein the side-looking expandable frame can maintain its rigidity when the angled internal catheter is steered.

19. The side-looking steering device of claim 16, wherein the angled internal catheter is bent at a 90-degree angle.

20. The side-looking steering device of claim 16, wherein the side-looking expandable frame comprises multiple leaflets, and the leaflets are joined by multiple struts.

21. A side-looking expandable frame for positioning an interventional device within a patient comprising:
a tubular base having a proximal end and a distal end, the tubular base sized for an angled internal catheter to extend therethrough;
a plurality of leaflets of varied lengths extending from the distal end of the tubular base; and
wherein each of the plurality of leaflets are biased to expand a first portion outwardly in a straight configuration and biased to expand a second portion outwardly at an angled configuration to a side.

22. The side-looking expandable frame of claim 21, wherein the angled configuration of the second portion differs from the straight configuration of the first portion by at least 50 degrees.

23. The side-looking expandable frame of claim 21, wherein the plurality of leaflets each vary in width.

24. The side-looking expandable frame of claim 21, wherein a distal end of the plurality of leaflets forms an eyelet configuration.

25. The side-looking expandable frame of claim 21, wherein the plurality of leaflets have distal ends with radiopaque markers.

26. The side-looking expandable frame of claim 21, wherein the plurality of leaflets have at least one strut.

27. The side-looking expandable frame of claim 26, wherein the at least one strut has a plurality of connection points, which allow for joining of the plurality of leaflets.

28. The side-looking expandable frame of claim 21, wherein the side-looking expandable frame is formed from a tube.

29. The side-looking expandable frame of claim 21, wherein the side-looking expandable frame is formed by laser cutting, photoetching, EDM, or by water jet abrasion.

30. The side-looking expandable frame of claim 21, wherein the side-looking expandable frame comprises a shape memory metal.

31. The side-looking expandable frame of claim 28, wherein the tube forming the side-looking expandable frame is asymmetric with one side having greater length than another.

32. The side-looking expandable frame of claim 28, wherein the tube is expanded to different diameters during a first shape setting, and once cooled, reheated to go through a second shape setting to create a plurality of shapes and angles.
